(19) United States
(12) Patent Application Publication
(10) Pub. No.: US 2022/0323046 A1
(43) Pub. Date: Oct. 13, 2022

(54) INTEGRATED SAMPLING AND TESTING DEVICE

(71) Applicants: Richard Fan Xie, San Diego, CA (US); Connie Yuxuan Jiang, San Diego, CA (US)

(72) Inventors: Richard Fan Xie, San Diego, CA (US); Connie Yuxuan Jiang, San Diego, CA (US)

(21) Appl. No.: 17/658,617

(22) Filed: Apr. 8, 2022

Related U.S. Application Data
(60) Provisional application No. 63/173,343, filed on Apr. 9, 2021.

(51) Int. Cl. A61B 10/00; G01N 33/569; B01L 3/00

(52) U.S. Cl. CPC A61B 10/0045; G01N 33/56983; B01L 3/502715; B01L 3/50273; G01N 2800/26; B01L 2200/04; B01L 2200/16; B01L 2300/0838; B01L 2300/123; B01L 2400/0478; B01L 2300/042

(57) ABSTRACT

Provided herein is an integrated sampling and testing device, comprising a sampling device that comprises a syringe and specimen collector, and a testing device that comprises a cap, cylindrical container, and adaptor configured for sampling or testing.

### CROSS REFERENCE TO RELATED APPLICATION

This application claims the benefit of the priority of U.S. Provisional Application No. 63/173,343, filed Apr. 9, 2021; the disclosure of which is incorporated herein by reference in its entirety.

### FIELD

Provided herein is an integrated sampling and testing device, comprising a sampling device that comprises a syringe and specimen collector, and a testing device that comprises a cap, cylindrical container, and adaptor configured for sampling or testing.

### BACKGROUND

Coronavirus disease 2019 (COVID-19) is caused by severe acute respiratory syndrome coronavirus-2 (SARS-CoV-2). Gorbalenya et al., Nat. Microbiol. 2020, 5, 536-44; Zhang et al., Science 2020, 368, 409-12. On Mar. 11, 2020, the World Health Organization declared COVID-19 a global pandemic. Zhang et al., Science 2020, 368, 409-12; Dai et al., Science 2020, 368, 1331-5. As of April 2021, there are over 100 million confirmed cases and close to three a million confirmed deaths globally. The COVID-19 pandemic poses as a great public health threat to the world. Early diagnosis of SARS-CoV-2 infection is crucial for ending the COVID-19 pandemic. Therefore, there is an urgent need for a safe and convenient device for a biological sample collection and testing.

### SUMMARY

Provided herein is a sampling device, comprising (i) a syringe having a bottom opening; and (ii) a specimen collector comprising a tube having top and bottom ends, and a sampling head attached to the bottom end of the tube; wherein the top end of the tube is attached to the bottom opening of the syringe; and the syringe is configured to release a fluid stored inside the syringe to the sampling head via the tube upon engagement.

Also provided herein is a sampling device comprising:

(i) a syringe comprising:
a syringe barrel having a top opening and a bottom;
a plunger; and
a contracted mouth;
wherein the plunger is slidably disposed via the top opening into the syringe barrel and the contracted mouth is located on the bottom of the syringe barrel; and

(ii) a specimen collector comprising:
a tube having top and bottom ends; and
a sampling head attached to the bottom end of the tube;

wherein the top end of the tube is attached to the contracted mouth of the syringe; and the syringe is configured to release a fluid stored inside the syringe to the sampling head via the tube upon engagement.

Additionally, provided herein is an integrated sampling and testing device, comprising:

(a) a sampling device comprising (i) a syringe having a bottom opening; and (ii) a specimen collector comprising a tube having top and bottom ends, and a sampling head attached to the bottom end of the tube; wherein the top end of the tube is attached to the bottom opening of the syringe; and the syringe is configured to release a fluid stored inside the syringe to the sampling head via the tube upon engagement; and

(b) a testing device comprising a cap; a cylindrical container having a top opening and a bottom; and an adaptor adjacent to the bottom of the cylindrical container and configured for sampling or testing;

wherein the cap is attached to the top opening of the cylindrical container to form an enclosure to enclose the sampling device within; and the cylindrical container is configured for receiving the sampling device with the sampling head fully disposed within, with the sampling head directed to the bottom of the cylindrical container.

Furthermore, provided herein is an integrated sampling and testing device, comprising:

(a) a sampling device comprising:
(i) a syringe comprising:
   a syringe barrel having a top opening and a bottom;
   a plunger; and
   a contracted mouth;
   wherein the plunger is slidably disposed via the top opening into the syringe barrel and the contracted mouth is located on the bottom of the syringe barrel; and
(ii) a specimen collector comprising:
   a tube having top and bottom ends; and
   a sampling head attached to the bottom end of the tube;
wherein the top end of the tube is attached to the contracted mouth of the syringe; and the syringe is configured to release a fluid stored inside the syringe to the sampling head via the tube upon engagement; and

(b) a testing device comprising a cap; a cylindrical container having a top opening and a bottom; and an adaptor located adjacent to the bottom of the cylindrical container and configured for sampling or testing;

wherein the cap is attached to the top opening of the cylindrical container to form an enclosure to enclose the sampling device within; and the cylindrical container is configured for receiving the sampling device with the sampling head fully disposed within, with the sampling head directed to the bottom of the cylindrical container.

### BRIEF DESCRIPTION OF THE DRAWINGS

FIG. 1 illustrates an exemplary sampling device 10 comprising a syringe 110 and a specimen collector 120; where the syringe 110 comprises a syringe barrel 111, a plunger 112, and a contracted mouth 113; where the specimen collector 120 comprises a tube 121 and a sampling head 122 (e.g., a swap); and where the syringe 110 is configured to release a fluid 115 stored in a fluid compartment 114 of the syringe 110 to the sampling head 122 via the tube 121 upon engagement.

FIG. 2 illustrates an exemplary application of a sampling device 10 of FIG. 1 for collecting a biological specimen from a human nasal cavity.

FIG. 3 is an exploded view of an exemplary integrated sampling and testing device 1, comprising a sampling device 10 of FIG. 1 and a testing device 20 that comprises a cylindrical container 211; an adaptor 212 configured for sampling or testing (e.g., a test strip holder for receiving a test strip 213); and a cap 220.

FIG. 4 is a cross-sectional view of an exemplary cap 220 of an integrated sampling and testing device 1 of FIG. 3, having an inner top surface and a bottom opening, and comprising a syringe pusher 221 configured for engagement with a plunger 112 of a syringe 110 of a sampling device 10, inner threads 222, and an extended lip 223.

FIG. 5 is a cross-sectional view of an exemplary cylindrical container 211 having an adaptor 212 (e.g., a test strip holder) attached to the outer side wall of the cylindrical container 211 close to the bottom; where the cylindrical container 211 comprises a fluid guide 217 as the bottom of the cylindrical container 211 and a hole 216 close to the bottom the cylindrical container 211.

FIG. 6 is a schematic view of an exemplary integrated sampling and testing device 1 with a cap 220 off and unengaged syringe 110, where the cylindrical container 211 comprises outer threads 214 next to the top opening of the cylindrical container 211, matching inner threads 222 of the cap 220.

FIG. 7 is a schematic view of an exemplary integrated sampling and testing device 1 with a cap 220 on and engaged syringe 110, where a fluid 115 stored inside a fluid compartment 114 of the syringe 110 has been transferred to a sampling head 122 and subsequently to the bottom of the cylindrical container 211; and where the cylindrical container 211 comprises a hole 216 (e.g., capillary hole) adjacent to the bottom of the cylindrical container 211, which is configured to allow a fluid in a reservoir 215 to pass through and reach a test strip 213 held in an adaptor 212 during testing.

### DETAILED DESCRIPTION

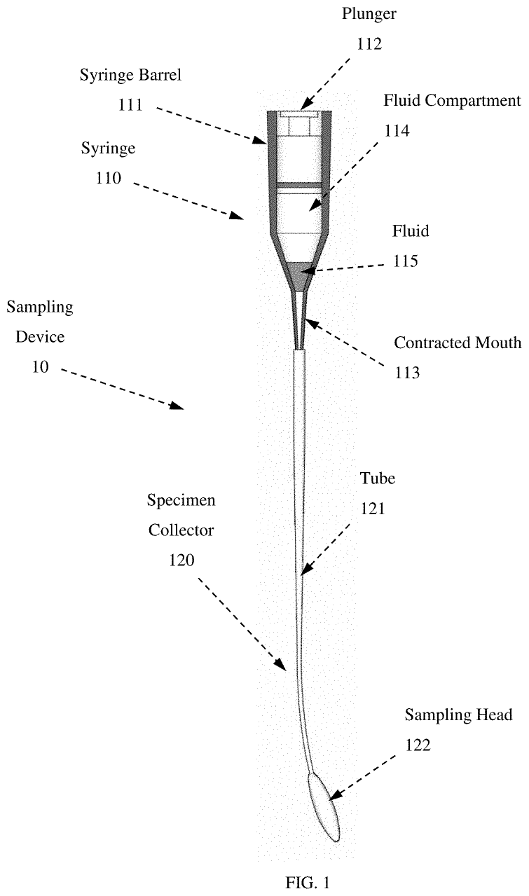

In one embodiment, as exemplified in FIG. 1, provided herein is a sampling device 10 comprising:

(i) a syringe 110 having a bottom opening; and

(ii) a specimen collector 120 comprising:
a tube 121 having top and bottom ends; and
a sampling head 122 attached to the bottom end of the tube 121;

wherein the top end of the tube 121 is attached to the bottom opening of the syringe 110; and the syringe 110 is configured to release a fluid 115 stored inside the syringe 110 to the sampling head 122 via the tube 121 upon engagement.

In another embodiment, as exemplified in FIG. 1, provided herein is a sampling device 10 comprising:

(i) a syringe 110 comprising:
a syringe barrel 111 having a top opening and a bottom;
a plunger 112; and
a contracted mouth 113;
wherein the plunger 112 is slidably disposed via the top opening into the syringe barrel 111 and the contracted mouth 113 is located on the bottom of the syringe barrel 111; and

(ii) a specimen collector 120 comprising:
a tube 121 having top and bottom ends; and
a sampling head 122 attached to the bottom end of the tube 121;

wherein the top end of the tube 121 is attached to the contracted mouth 113 of the syringe 110; and the syringe 110 is configured to release a fluid 115 stored inside the syringe 110 to the sampling head 122 via the tube 121 upon engagement.

In certain embodiments, the contracted mouth 113 is made of an elastic material. In certain embodiments, the contracted mouth 113 is made of silicone. In certain embodiments, the contracted mouth 113 has two states: close and open. In certain embodiments, the contracted mouth 113 is close when the syringe 110 is not engaged, thus not allowing a fluid 115 inside the fluid compartment 114 of the syringe 110 to flow out. In certain embodiments, the contracted mouth 113 is open upon the engagement of the syringe 110, thus allowing a fluid 115 inside the fluid compartment 114 of the syringe 110 to flow out.

In certain embodiments, the sampling head 122 is a swab. In certain embodiments, the sampling head 122 is a nasal swab. In certain embodiments, the sampling head 122 is a throat swab. In certain embodiments, the sampling head 122 is a nasopharyngeal swab. In certain embodiments, the sampling head 122 is a flocked swab. In certain embodiments, the sampling head 122 is a swab made of cotton, polyester, nylon, or flocked nylon. In certain embodiments, the sampling head 122 is a swab made of cotton. In certain embodiments, the sampling head 122 is a swab made of polyester. In certain embodiments, the sampling head 122 is a swab made of nylon. In certain embodiments, the sampling head 122 is a swab made of flocked nylon.

In certain embodiments, the syringe 110 contains a fluid 115 in a predetermined amount in the fluid compartment 114 of the syringe 110. In certain embodiments, the syringe 110 contains a fluid 115 in an amount ranging from about 0.1 mL to about 20 mL, from about 0.5 mL to about 10 mL, or from about 1 mL to about 5 mL. In certain embodiments, the syringe 110 contains a fluid 115 in an amount ranging from about 0.1 mL to about 20 mL. In certain embodiments, the syringe 110 contains a fluid 115 in an amount ranging from about 0.5 mL to about 10 mL. In certain embodiments, the syringe 110 contains a fluid 115 in an amount ranging from about 1 mL to about 5 mL. In certain embodiments, the fluid 115 is an aqueous solution. In certain embodiments, the fluid 115 is a buffer solution. In certain embodiments, the fluid 115 is water or saline.

In certain embodiments, the syringe 110 comprises two or more fluid compartments, each for storing a fluid. In certain embodiments, the syringe 110 comprises two or more fluid compartments, each for storing a different fluid. In certain embodiments, the syringe 110 comprises two fluid compartments, each for storing a fluid. In certain embodiments, the syringe 110 comprises two fluid compartments, each for storing a different fluid. In certain embodiments, the two or more fluid compartments are configured in such a way that the fluids from different compartments do not mix until flowing out of the contracted mouth 113 after the syringe is engaged.

In certain embodiments, the fluid compartment 114 of the syringe 110 is divided into two or more subcompartments, each for storing a fluid. In certain embodiments, the fluid compartment 114 of the syringe 110 is divided into two or more subcompartments, each for storing a different fluid. In certain embodiments, the fluid compartment 114 of the syringe 110 is divided into two subcompartments, each for storing a fluid. In certain embodiments, the fluid compartment 114 of the syringe 110 is divided into two subcompartments, each for storing a different fluid. In certain embodiments, the two or more subcompartments are configured in such a way that the fluids from different subcompartments do not mix until flowing out of the contracted mouth 113 after the syringe is engaged.

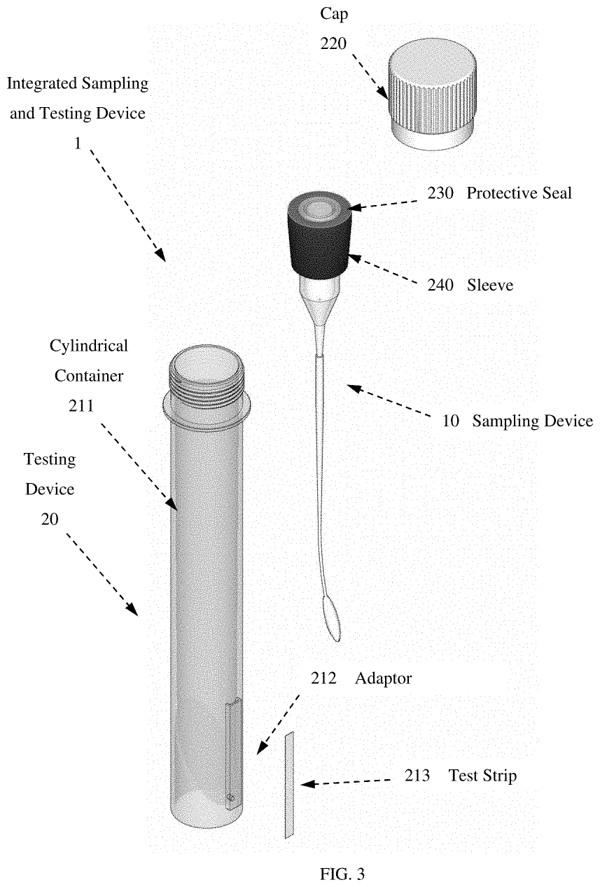

In certain embodiments, the syringe 110 further comprises a protective seal 230 as illustrated in FIG. 3, which is configured to cover the plunger 112 on the top of the syringe 110 to prevent the plunger 112 from accidental engagement. In certain embodiments, the protective seal 230 is made of a plastic or foil. In certain embodiments, the protective seal 230 is made of a plastic. In certain embodiments, the protective seal 230 is made of a foil. In certain embodiments, the protective seal 230 is made of a tinfoil.

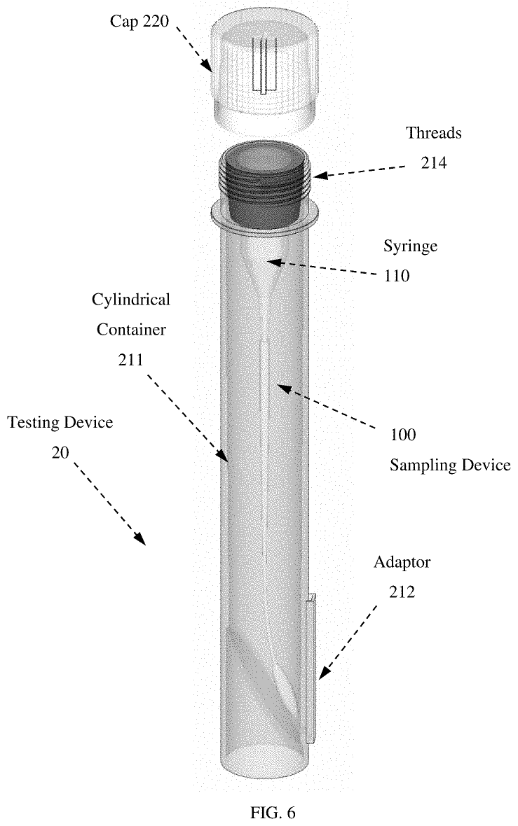

In certain embodiments, the syringe 110 further comprises a sleeve 240 surrounding the syringe barrel 111. In certain embodiments, the sleeve 240 is made of an elastic material. In certain embodiments, the sleeve 240 is made of rubber. In certain embodiments, as illustrated in FIGS. 3 and 6, the sleeve 240 functions as a stopper to secure a sampling device 10 within a cylindrical container 211 of a testing device 20.

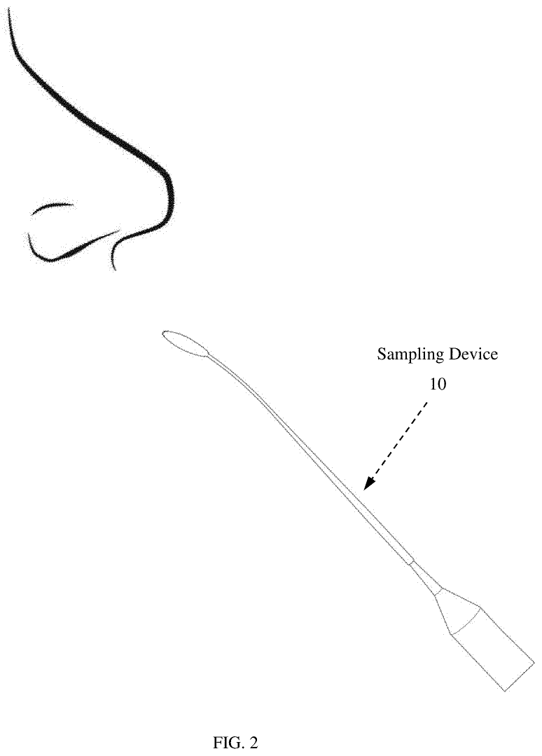

FIG. 2 illustrates an exemplary application of a sampling device 10 provided herein for collecting a biological sample from a human nasal cavity, where the sampling head 122 (e.g., a swap) of the sampling device 10 is inserted into a human nostril, reaching into the nasal cavity to collect nasal mucus.

In certain embodiments, the specimen collector 120 is suitable for collecting a biological specimen. In certain embodiments, the specimen collector 120 is suitable for collecting a bodily fluid. In certain embodiments, the specimen collector 120 is suitable for collecting a blood, cerebrospinal fluid, feces, saliva, serum, sputum, or urine sample. In certain embodiments, the specimen collector 120 is suitable for collecting an environmental specimen.

In certain embodiments, the specimen collector 120 is used without being attached to the syringe 110, and after specimen collection, the specimen collector 120 is reattached to a syringe 110. In certain embodiments, the sampling device 10 is suitable for field specimen collection.

In yet another embodiment, as exemplified in FIG. 3, provided herein is an integrated sampling and testing device 1, comprising a sampling device 10 provided herein; and a testing device 20.

In one embodiment, as exemplified in FIG. 3, provided herein is an integrated sampling and testing device 1, comprising:

(a) a sampling device 10 comprising (i) a syringe 110 having a bottom opening; and (ii) a specimen collector 120 comprising a tube 121 having top and bottom ends, and a sampling head 122 attached to the bottom end of the tube 121; wherein the top end of the tube 121 is attached to the bottom opening of the syringe 110; and the syringe 110 is configured to release a fluid 115 stored inside the syringe 110 to the sampling head 122 via the tube 121 upon engagement; and

(b) a testing device 20 comprising a cap 220; a cylindrical container 211 having a top opening and a bottom; and an adaptor 212 adjacent to the bottom of the cylindrical container 211;

wherein the cap 220 is attached to the top opening of the cylindrical container 211 to form an enclosure to enclose the sampling device 10 within; and the cylindrical container 211 is configured for receiving the sampling device 10 with the sampling head 122 fully disposed within, with the sampling head 122 directed to the bottom of the cylindrical container 211.

In another embodiment, as exemplified in FIG. 3, provided herein is an integrated sampling and testing device 1, comprising:

(a) a sampling device 10 comprising:
(i) a syringe 110 comprising:
   a syringe barrel 111 having a top opening and a bottom;
   a plunger 112; and
   a contracted mouth 113;
   wherein the plunger 112 is slidably disposed via the top opening into the syringe barrel 111 and the contracted mouth 113 is located on the bottom of the syringe barrel 111; and
(ii) a specimen collector 120 comprising:
   a tube 121 having top and bottom ends; and
   a sampling head 122 attached to the bottom end of the tube 121;
wherein the top end of the tube 121 is attached to the contracted mouth 113 of the syringe 110; and the syringe 110 is configured to release a fluid 115 stored inside the syringe 110 to the sampling head 122 via the tube 121 upon engagement; and

(b) a testing device 20 comprising a cap 220; a cylindrical container 211 having a top opening and a bottom; and an adaptor 212 adjacent to the bottom of the cylindrical container 211;

wherein the cap 220 is attached to the top opening of the cylindrical container 211 to form an enclosure to enclose the sampling device 10 within; and the cylindrical container 211 is configured for receiving the sampling device 10 with the sampling head 122 fully disposed within, with the sampling head 122 directed to the bottom of the cylindrical container 211.

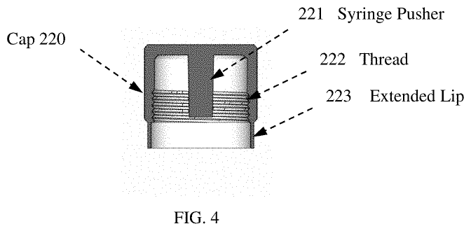

In certain embodiments, as exemplified in FIG. 4, the cap 220 having a top inner surface and a bottom opening comprises a syringe pusher 221 attached to the top inner surface of the cap 220, inner threads 222, and an extended lip 223. In certain embodiments, the syringe pusher 221 is attached to the center of the top inner surface of the cap 220 and extends toward the bottom opening of the cap 220. In certain embodiments, the syringe pusher 221 is a pole protruding downward from the inner top surface to the bottom opening of the cap 220. In certain embodiments, the syringe pusher 221 is cylindrical or quadrangular. In certain embodiments, the syringe pusher 221 has a sharp or pointy head that can penetrate a protective seal 230 preventing the plunger 112 of the syringe 110 from accidental engagement. During operation, the cap 220 is rotated in a downward closing motion onto the top of the cylindrical container 211, causing the pusher 221 to break the protective seal 230 and then push the plunger 112 of the syringe 110 to release a fluid 115 from the fluid compartment 114 of the syringe 110 to the sampling head 122 via the tube 121. In certain embodiments, the inner threads 222 of the cap 220 engage with outer threads 214 adjacent to the top opening of the cylindrical container 211. In certain embodiments, the cap 220 and the cylindrical container 211 are threadedly connected with each other. In certain embodiments, the extended lip 223 is configured to work together with a flange adjacent to the top end of the cylindrical container 211 to prevent the cap 220 from being overtighten.

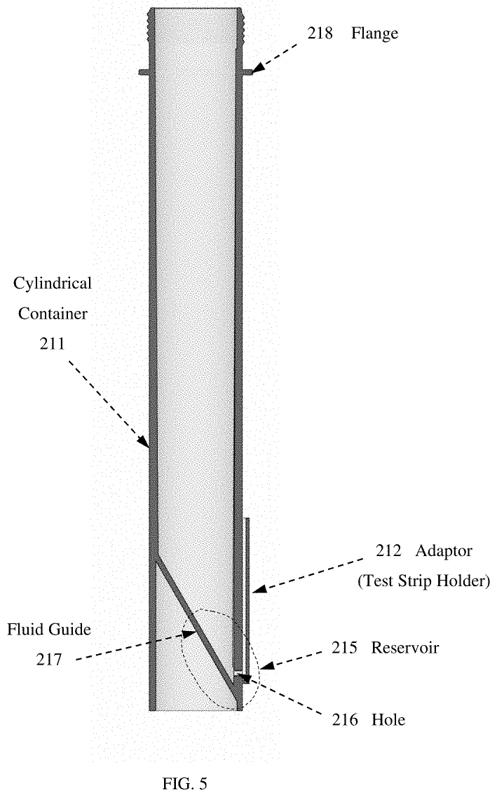

In certain embodiments, the sleeve 240 of the syringe 120 functions as a stopper to securely “plug” the sampling device 10 into the cylindrical container 211. In certain embodiments, as exemplified in FIG. 5, the cylindrical container 211 further comprises outer threads 214 adjacent to the top opening of the cylindrical container 211, where the outer threads 214 are configured to match the inner threads 222 of the cap 220. In certain embodiments, as exemplified in FIG. 4, the cylindrical container 211 further comprises a flange 218 adjacent to the top opening of the cylindrical container 211, where the flange 218 and extended lip 223 are configured to work together to prevent the cap 220 from being overtighten. As exemplified in FIG. 5, where the cylindrical container 211 comprises outer threads 214 and a flange 218, the outer threads 214 are closer to the top opening of the cylindrical container 211 than the flange 218. In certain embodiments, as exemplified in FIG. 5, the cylindrical container 211 further comprises a fluid guide 217 as a bottom of the cylindrical container 211 for guiding the fluid to the adaptor 212 via a hole 216. In certain embodiments, as exemplified in FIG. 4, the fluid guide 217 is tilted relative to the side wall of the cylindrical container 211. In certain embodiments, as exemplified in FIG. 5, the cylindrical container 211 further comprises a hole 216 adjacent to the bottom of the cylindrical container 211 and configured for sampling or testing. In certain embodiments, the hole 216 is a capillary hole configured for testing, where the capillary hole 216 creates a fluidic connection between a reservoir 215 and the adaptor 212. When the adaptor 212 is a test strip holder, the capillary hole 216 allows the fluid in the reservoir 215 at the bottom of the cylindrical container 211 to flow into the test strip holder 212 and to get into contact with a test strip 213.

In certain embodiments, as exemplified in FIG. 5, the adaptor 212 is attached to the outer side wall and adjacent to the bottom of the cylindrical container 211. In certain embodiments, as exemplified in FIG. 5, the adaptor 212 is a test strip holder. In certain embodiments, the adaptor 212 comprises a window for visualization of a test result.

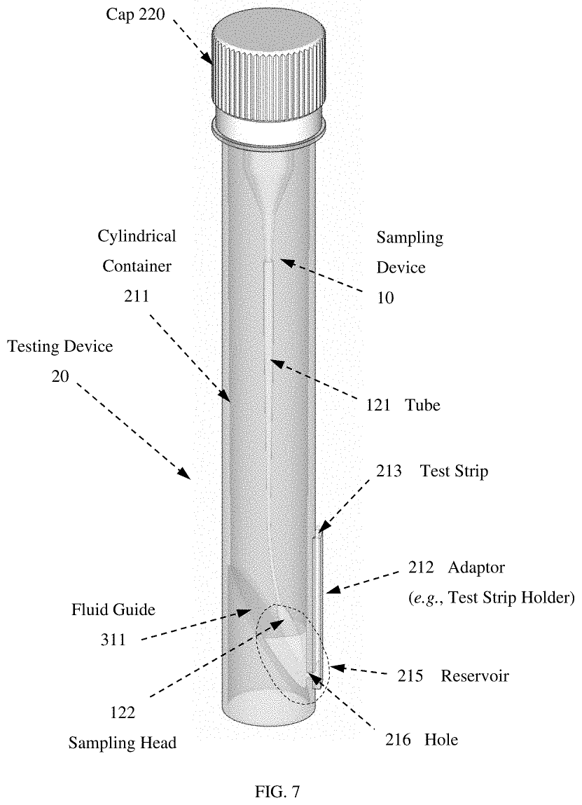

FIG. 6 is a schematic view of an exemplary integrated sampling and testing device 1 with the cap 220 off and unengaged syringe 110, where a fluid 115 is stored inside a fluid compartment 114 of the syringe 110. FIG. 7 is a schematic view of an exemplary integrated sampling and testing device 1 with the cap 220 on and engaged syringe 110, where a fluid 115 released from the fluid compartment 114 of the syringe 110 flows to the sampling head 122 and subsequently to the bottom of the cylindrical container 211.

In operation, after a sampling device 10 collects a specimen, the sample device 10 is inserted securely into a cylindrical container 211 of a testing device 20 and a cap 220 is screwed onto the top opening of the cylindrical container 211 of the testing device 20. As the cap 220 is being secured onto the top opening of the cylindrical container 211, the syringe pusher 221 breaks the protective seal 230, engages with the plunger 112 of the syringe 110, and pushes the fluid 115 stored inside the syringe 110 out of the contracted mouth 113 to the sampling head 122 via the tube 121, and washes the specimens collected on the sampling head 122 into the bottom of the cylindrical container 211 of the testing device 20. In certain embodiments, the amount of the fluid released from the syringe 110 is controlled by the number of rotations of the cap 320 during closing.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A sampling device, comprising (i) a syringe having a bottom opening; and (ii) a specimen collector comprising a tube having top and bottom ends, and a sampling head attached to the bottom end of the tube; wherein the top end of the tube is attached to the bottom opening of the syringe; and the syringe is configured to release a fluid stored inside the syringe to the sampling head via the tube upon engagement.

2. The sampling device of claim 1, comprising:
(i) a syringe comprising:
a syringe barrel having a top opening and a bottom;
a plunger; and
a contracted mouth;
wherein the plunger is slidably disposed via the top opening into the syringe barrel and the contracted mouth is located on the bottom of the syringe barrel; and
(ii) a specimen collector comprising:
a tube having top and bottom ends; and
a sampling head attached to the bottom end of the tube;
wherein the top end of the tube is attached to the contracted mouth of the syringe; and the syringe is configured to release a fluid stored inside the syringe to the sampling head via the tube upon engagement.

3. The sampling device of claim 2, wherein the plunger is fully disposed within the syringe barrel.

4. The sampling device of claim 2, wherein the contracted mouth is made of an elastic material or wherein the contracted mouth is made of silicone.

5. The sampling device of claim 1, wherein the collector head is a swab.

6. The sampling device of claim 5, wherein the swab is a nasal swab, a throat swab, or a nasopharyngeal swab; or wherein the swab is a flocked swab.

7. The sampling device of claim 5, wherein the swab is made of an absorbing material; or wherein the swab is made of cotton, polyester, nylon, or flocked nylon.

8. The sampling device of claim 1, wherein the syringe is detachable from the specimen collector.

9. The sampling device of claim 2, wherein the syringe further comprises a protective seal configured to cover the plunger on the top to prevent the plunger from accidental engagement.

10. The sampling device of claim 9, wherein the protective seal is made of a plastic material or a foil.

11. The sampling device of claim 2, wherein the syringe further comprises a sleeve surrounding the syringe barrel.

12. The sampling device of claim 11, wherein the sleeve is made of an elastic material; or wherein the sleeve is made of rubber.

13. An integrated sampling and testing device, comprising:
(a) a sampling device of claim 1; and
(b) a testing device comprising a cap; a cylindrical container having a top opening and a bottom; and an adaptor adjacent to the bottom of the cylindrical container and configured for sampling or testing;
wherein the cap is attached to the top opening of the cylindrical container to form an enclosure to enclose the sampling device within; and the cylindrical container is configured for receiving the sampling device with the sampling head fully disposed within, with the sampling head directed to the bottom of the cylindrical container.

14. The integrated sampling and testing device of claim 13, wherein the cap having a top inner surface and a bottom opening comprises a syringe pusher attached to the top inner surface of the cap, inner threads, and an extended lip.

15. The integrated sampling and testing device of claim 14, wherein a syringe pusher is configured to break the protective seal covering the plunger and to engage the syringe to release a fluid to the sampling head via the tube.

16. The integrated sampling and testing device of claim 13, wherein the sleeve surrounding the syringe barrel of the sampling device functions as a stopper to secure the sampling device within the cylindrical container of the testing device.

17. The integrated sampling and testing device of claim 13, wherein the cylindrical container further comprises a fluid guide as a bottom of the cylindrical container.

18. The integrated sampling and testing device of claim 13, wherein the cylindrical container further comprises a hole adjacent to the bottom of the cylindrical container and configured for sampling or testing.

19. The integrated sampling and testing device of claim 18, wherein the hole is a capillary hole.

20. The integrated sampling and testing device of claim 13, wherein the adaptor is a test strip holder.
